(19) United States
(12) Patent Application Publication
(10) Pub. No.: US 2013/0210907 A1
(43) Pub. Date: Aug. 15, 2013

(54) METHOD FOR PREVENTING OR TREATING ENTEROCHROMAFFIN CELL HYPERPLASIA-RELATED DISEASES BY QUERCETIN ADMINISTRATION

(71) Applicant: HONG KONG BAPTIST UNIVERSITY, (US)

(72) Inventors: Zhaoxiang BIAN, Hong Kong (HK); Hongyan QIN, Hong Kong (HK); Haitao XIAO, Hong Kong (HK); Albert Sun-Chi CHAN, Hong Kong (HK); Hongxi XU, Hong Kong (HK); Shilin CHEN, Hong Kong (HK); Dajian YANG, Hong Kong (HK)

(73) Assignee: HONG KONG BAPTIST UNIVERSITY, Hong Kong (HK)

(21) Appl. No.: 13/767,865

(22) Filed: Feb. 14, 2013

Related U.S. Application Data
(60) Provisional application No. 61/598,926, filed on Feb. 15, 2012.

(51) Int. Cl. A61K 31/353; A23L 1/30

(52) U.S. Cl. CPC A61K 31/353; A23L 1/3002
USPC 514/456; 549/400

(57) ABSTRACT

The present invention provides methods and compositions for preventing or treating enterochromatffin cell hyperplasia-related diseases. The compositions comprise a therapeutic effective amount of Quercetin, glycosylated Quercetin, or mixture thereof. The methods comprises administering said composition as well as preparing said composition as a pharmaceutical formulation,

### CROSS-REFERENCE TO RELATED APPLICATIONS

The present application claims priority of U.S. provisional application No. 61/598,926 filed Feb. 15, 2012, and which the disclosure is hereby incorporated by reference.

### FIELD OF INVENTION

The present invention relates to a method of preventing or treating enterochromaffin cell hyperplasia-related diseases such as inflammatory bowel disease, irritable bowel syndrome and irritable pouch syndrome in mammals, which comprises administering an effective amount of Quercetin.

### BACKGROUND OF INVENTION

It has been reported that enterochromaffin (EC) cell hyperplasia and serotonin (5-HT) hyperactivity are closely related to various kind of diseases such as inflammatory bowel disease (IBD), irritable bowel syndrome (IBS) and irritable pouch syndrome (IPS), especially post-infectious varieties of such diseases. EC cell, the major enteroendocrine cell type in the gastrointestinal tract, is responsible for the synthesis, storage and release of 5-HT in the gut, and it is reported that about 90% of the total 5-HT in the body is found in EC cells. The rate-limiting enzyme controlling 5-HT synthesis is tryptophan hydroxylase (TPH), which produces 5-HT by using the dietary amino acid L-tryptophan. In addition, EC cells have been regarded as the intestinal sensors that detect chemical, mechanical or pathological stimuli in the lumen. 5-HT can be quickly released from EC cells following mucosal stimulation, and activated serotonergic receptors located on nerve fibers. The actions of released 5-HT are terminated by uptake of the 5-HT via serotonin reuptake transporter into the enterocytes. As a neurotransmitter, 5-HT modulates visceral sensation and motor function through influencing the sympathetic, parasympathetic and enteric nerves systems. Nowadays, several studies, as reported in Shen B, Liu W, Remzi F H, Shao Z, Lu H, DeLaMotte C, et al. Enterochromaffin cell hyperplasia in irritable pouch syndrome. Am J. Gastroenterol. 2008 September; 103(9):2293-300, Dunlop S P, Jenkins D, Neal K R, Spiller R C. Relative importance of enterochromaffin cell hyperplasia, anxiety, and depression in postinfectious IBS. Gastroenterology. 2003 December; 125(6):1651-9, Spiller R C, Jenkins D, Thornley J P, Hebden J M, Wright T, Skinner M, et al. Increased rectal mucosal enteroendocrine cells, T lymphocytes, and increased gut permeability following acute Campylobacter enteritis and in post-dysenteric irritable bowel syndrome. Gut. 2000 December; 47(6):804-11 and Mawe G M, Collins S M, Shea-Donohue T. Changes in enteric neural circuitry and smooth muscle in the inflamed and infected gut. Neurogastroenterol Motil. 2004 April; 16 Suppl 1:133-6, have showed that EC cell hyperplasia and 5-HT hyperactivity played an important role in the symptoms development of many gastrointestinal diseases, such as IBD, IBS, and IPS.

EC cells originate from multi-potent intestinal stem cells located near the base of the crypts; after proliferation and differentiation, they continue to mature and migrate up the villous tips where they are extruded eventually. With the continuous turnover, the lost EC cell must be replaced by a newly formed EC cell in order to maintain the homeostasis. Nowadays, it is generally accepted that the normal enteroendocrine cells are a population of terminally differentiated, non-proliferating cells. However, it is reported that EC cells can retain a small capacity of proliferation under stimulation, for example, EC-like cells in the stomach possessed self-replicative capacity after stimulated by lipopolysaccharide. To date, the pathogenesis mechanism of EC cell hyperplasia in PI-IBS is unknown, but it is proposed in Dunlop S P, Jenkins D, Neal K R, Spiller R C. Relative importance of enterochromaffin cell hyperplasia, anxiety, and depression in postinfectious IBS. Gastroenterology. 2003 December; 125(6):1651-9 that the increased EC cell numbers might arise from increased division from stem cells, increased EC cell progenitors, or decreased EC cell apoptosis. In vitro study of EC cells seems difficult, for its relatively small proportion (˜1%) in epithelia cells, and there are about fifteen other types of enteroendocrine cells with similar structure properties. Therefore, certain cell lines, which derived from human tumors and presented the features of 5-HT synthesis, storage and release, have been utilized as EC cell models. Currently, the commonly used EC cell models, such as those reported in Doihara H, Nozawa K, Kojima R, Kawabata-Shoda E, Yokoyama T, Ito H. QGP-1 cells release 5-HT via TRPA1 activation; a model of human enterochromaffin cells. Mol Cell Biochem. 2009 November; 331(1-2):239-45, Kidd M, Modlin I M, Gustafsson B I, Drozdov I, Hauso O, Pfragner R. Luminal regulation of normal and neoplastic human EC cell serotonin release is mediated by bile salts, amines, tastants, and olfactants. Am J Physiol-Gastr L. 2008 August; 295(2):G260-G72, Kim M S, Cooke H J, Javed N H, Carey H V, Christofi F, Raybould H E. D-glucose releases 5-hydroxytryptamine from human BON cells as a model of enterochromaffin cells. Gastroenterology. 2001 December; 121(6): 1400-6 and Christofi F L, Kim M, Wunderlich J E, Xue J J, Suntres Z, Cardounel A, et al. Endogenous adenosine differentially modulates 5-hydroxytryptamine release from a human enterochromaffin cell model. Gastroenterology. 2004 July; 127(1):188-202, are come from the human carcinoid tumours of pancreas or small intestine, such as BON cell, KRJ-1 cell, and QGP-1 cell.

It is proposed that any alterations in 5-HT availability, such as biosynthesis, release, or uptake, may contribute to the disordered gastrointestinal sensation and motility; therefore, therapeutic strategies based on 5-HT availability have been studied extensively. Nowadays, the inhibitor of 5-HT synthesis enzyme, 5-HT₃ receptor antagonists, and 5-HT₄ receptor agonists have been investigated and applied clinically to relieve symptoms of gastrointestinal disorders, such as IBS. Alosetron, a representative antagonist of 5-HT₃ receptors, is an effective agent in the symptoms improvement of D-IBS, but the serious adverse effects (i.e. constipation and ischemic colitis) made it only being used under restrictive guideline. Tegaserod, a selective partial agonist of the 5-HT₄ receptor, improved bowel habit but not abdominal pain in IBS patients, but the possible cardiovascular adverse effects made it being withdrawn. Despite the notable adverse events associated with these agents, strategies targeting serotonergic systems remain active even though they are mainly found to relieve symptoms. Knowing that EC cell is the major sources of 5-HT synthesis and release, the number and function of EC cells arouse much attention, but up to now, little therapeutic strategy has been found to prevent or treat EC cell hyperplasia.

Quercetin, a flavonoid with low-molecular weight, is widely distributed in plant food (i.e. fruit, vegetables, tea, nuts, seeds, flowers, et al.) and herbal medicines (i.e. Ginkgo biloba, hypericum perforatum, Sambucus Canadensis, et al.). Quercetin exists normally as glycol-conjugated compound, and its glycosylated forms are presented by different monosaccharides it conjugated, such as hyperoside, isoquercetin, quercetrin, rutin, spiraeodide, and troxerutin (refer Table 1). High quantities of Quercetin glycosides are found in the diet, and most of these glycosyl groups of Quercetin can be released after ingestion by the bacteria existed in mouth and gut. It is reported that the epithelial cells in gastrointestinal tract are likely to be exposed to higher local concentrations for the low bioavailability of Quercetin, which may increase the action in the gut. Quercetin is firstly considered as a natural mast cell stabilizer, as its structure is very similar to disodium cromoglycate and showed potent inhibitory effect on mast cell secretion. Nowadays, a wide range of biological activities of Quercetin, i.e. anti-inflammation, antioxidation, anti-cancer and analgesic effect, have been widely reported, which is believed to provide beneficial effects on human health. Further, in spite of the potent efficacies, Quercetin exhibits no toxicity when it is orally administered to humans at a dose of 1000 mg/kg/day for several months.

TABLE 1

| Flavono Glycoside | Aglycone Form |
|---|---|
| Hyperoside | Quercetin |
| Isoquercetin | Quercetin |
| Quercetrin | Quercetin |
| Rutin | Quercetin |
| Spiraeoside | Quercetin |
| Troxerutin | Quercetin |

U.S. patent application Ser. No. 11/510, 152, filed Aug. 24, 2006) discloses that the small molecule inhibitors of the chloride-ion channel, such as Quercetin, can be used to treat symptoms of diarrhea-predominant IBS by inhibiting secretion of chloride ions. Another U.S. patent application Ser. No. 09/056,707, filed Apr. 8, 1998) describes that many flavonoids are inhibitors of mast cell secretion, which can be used to treat atopic allergic diseases, such as IBD. These inventions describe the methods for the treatment of IBS or IBD either by inhibiting ions secretion or by inhibiting mast cell secretion, and these methods are all involved Quercetin. Nonetheless, these prior arts only treat the symptoms associated with IBS or IBD after the onset of such diseases. Concerning the important role of EC cell hyperplasia and 5-HT hyperactivity in the pathogenesis of many diseases, there remains a strong need to provide effective method to treat EC cell hyperplasia related diseases, and prevent such diseases as IBS, IBD, and IPS, especially their post-infectious varieties, and some other guts disorders such as—untreated coeliac diseases and the chronic gastritis of pernicious anaemia, before their onset. Moreover, there is an association between gastric endocrine cell proliferation and chronic, sustained hypergastrinemia diseases. Differ from above listed inventions, this present invention provides a new method for treating or preventing such EC cell hyperplasia-related diseases.

The present inventors have endeavored to develop a novel pharmacological use of bioflavonoids, which are abundantly presented in fruit, vegetables, herbs, and foodstuffs. As a result, it has been discovered that Quercetin is effective in treating or preventing EC cell hyperplasia-related diseases. Specifically, Quercetin can significantly attenuate visceral hyperalgesia; reduce colonic EC cell number, 5-HT content, and TPH expression; attenuate mechanical stimuli-induced 5-HT release in a rat model of post-infectious irritable bowel syndrome (PI-IBS). It is notable that Doxantrazole, a mast cell stabilizer, did not show any effect on EC cell hyperplasia, even though it also has analgesic effect in PI-IBS rats. Moreover, Quercetin is also found to inhibit cell proliferation and induce cell apoptosis in an EC cell model. All these data indicated that Quercetin can be used to treat EC cell hyperplasia, and the underlying mechanisms may be mediated by inhibiting EC cell proliferation and inducing EC cell apoptosis.

Citation or identification of any reference in this section or any other section of this application shall not be construed as an admission that such reference is available as prior art for the present application.

### SUMMARY OF INVENTION

Accordingly, it is an object of the present invention to provide a method for treating or preventing enterochromaffin cell hyperplasia-related diseases, such as inflammatory bowel disease, irritable bowel syndrome and irritable pouch syndrome in a mammal.

Accordingly, it is a primary object of the present invention to provide a method of treating or preventing enterochromaffin cell hyperplasia-related diseases in a mammal.

In accordance with one aspect of the present invention, there is provided a method for treating or preventing enterochromaffin cell hyperplasia-related diseases, which comprises administering thereto an effective amount of Quercetin, glycosylated Quercetin or a mixture thereof.

In one embodiment of the present invention Quercetin exerts therapeutic effect on enterochromaffin cell hyperplasia-related diseases such as inflammatory bowel disease, irritable bowel syndrome and irritable pouch syndrome.

In another embodiment of the present invention a pharmaceutical formulation may be prepared in accordance with any of the conventional procedures. In preparing the formulation, the active ingredient is preferably admixed or diluted with a carrier, or enclosed within a carrier, which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, power, sachet, elixir, suspension, emulsion, solution, syrup, aerosol, soft and hard gelatin capsule, sterile injectable solution, sterile packaged powder and the like.

In a further embodiment of the present invention the pharmaceutical composition of the present invention contains the active ingredient in an amount ranging from 0.01 to 100 wt %. Further, the pharmaceutical composition of the present invention can be administered via various routes including oral, transdermal, subcutaneous, intravenous and intramuscular introduction. However, it should be understood that the amount of the active ingredient actually administered would be apparent to those skilled in the art in light of various relevant factors including the condition to be treated, the chosen route of administration, the age, sex and body weight of the individual patient, and the severity of the patient's symptom; and therefore, the above dose should not be intended to limit the scope of the invention in any way.

Moreover, in yet another embodiment of the present invention Quercetin can be advantageously incorporated in foods or, beverages for the purpose of treating or preventing inflammatory bowel disease, irritable bowel syndrome, and irritable pouch syndrome.

As described above, it is an embodiment of the present invention that Quercetin can be used as an effective, non-side effect pharmaceutical agent for treating or preventing enterochromaffin cell hyperplasia-related disease such as inflammatory bowel disease, irritable bowel syndrome, and irritable pouch syndrome.

Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the representative EC cell staining in colon mucosa of (A) normal rats, (B) PI-IBS rats, (C) Doxantrazole treated rats, and (D) Quercetin (10 mg/kg) treated rats (Scale bars, 200 μm).

FIG. 2 shows the representative electron microsgraphs of EC cells (×8900) and their secretory granules (×10000) from the colonic mucosa of normal rats, PI-IBS rats, and Querctetin (10 mg/kg) treated rats.

FIG. 3 shows the effect of Quercetin on viability of QGP-1 cells. Data are represented as mean±SD of three determinations.

FIG. 4 shows the Quercetin induced cytotoxicity. Cytotoxicity of Quercetin is evaluated by lactate dehydrogenase (LDH) release from Quercetin-treated QGP-1 cells.

FIG. 5 show the effects of Quercetin on cell cycle regulation in QGP-1 cells. Panel (FIG. 5A) shows the representative flow cytometric analysis results. Panel (FIG. 5B) depicts the western immunoblots and statistical analysis of altered proteins expression.

FIG. 6 shows the effects of Quercetin on cell apoptosis in QGP-1 cells. Panel (A) shows the representative DAPI staining in QGP-1 cells treated with 0.1% DMSO (A1) and 100 μM Quercetin (A2). Apoptosis cells are characterized by condensed nuclear (arrow) or fragmented nuclear chromatin (arrowhead; scale bar, 20 μm). Statistical analyses of apoptosis rate and sub-G0/G1 fraction are shown in panel (B) and (C), respectively.

### DETAILED DESCRIPTION OF INVENTION

The present invention is not to be limited in scope by any of the specific embodiments described herein. The following embodiments are presented for exemplification only.

Throughout the specification, the term “enterochromaffin cell” designates a endocrine cell type occurring in the epithelia lining of the gut and the respiratory tract that mainly synthesize and release serotonin.

The term “enterochromaffin cell hyperplasia” means increased enterochromaffin cell number or enlarged enterochromaffin cell size than normal level. Enterochromaffin cell hyperplasia in ordinarily considered to be harmful to health.

The term “enterochromaffin cell hyperplasia-related disease” means a disease which is caused by a increased enterochromaffin cell number or enlarged enterochromaffin cell size, and/or a disease whose symptoms include enterochromaffin cell hyperplasia. Examples of such a disease include, but are not limited to, inflammatory bowel disease, irritable bowel syndrome, irritable pouch syndrome and the like.

The present invention provide a method of treating enterochromaffin (EC) cell hyperplasia-related diseases comprising administering a composition comprises a therapeutic effective amount of Quercetin, glycosylated Quercetin or mixture thereof. Quercetin is 2-(3,4-dihydroxyphenyl)-3,5,7-trihydroxy-4H-chromen-4-one having a chemical structure of:

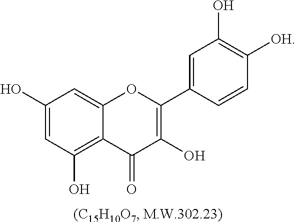

Specifically, the present invention provides treatment and prevention of enterochromaffin cell related diseases and conditions induced by infections. In one embodiment, the enterochromaffic cell related diseases and conditions that can be treated or prevented by the present invention develop after exposure of infection. The infection is caused by enteric pathogens. For example: Campylobacter species, Salmonella species, diarrheagenic strains of Escherichia coli, Shigella species and Trichinella species. Examples of cell hyperplasia-related diseases treated and prevented by the present invention before onset thereof includes, but are not limited to, inflammatory bowel disease, irritable bowel syndrome and irritable pouch syndrome, post-infectious inflammatory bowel disease, post-infectious irritable bowel syndrome, post-infectious irritable pouch syndrome, untreated coeliac diseases, chronic gastritis of pernicious anaemia and other gastrointestinal disorders or conditions induced by infections. Moreover, the present invention is highly specific as the present invention exhibits no effects on enterochromaffin cell of healthy normal individuals.

Quercetin, glycosylated Quercetin of the present invention may be extracted from various plants including vegetables such as onion and tomato, fruits such as apples and grapes, herbs such as Ginkgo biloba, hypericum perforatum, and Sambucus Canadensis, and grains such as buckwheat, or synthesized in accordance with the conventional process. Glycosylated Quercetin of the present invention includes, but is not limited to, hyperoside, isoquercetin, quercetrin, rutin, spiraeodide, and troxerutin. One skilled in the art would readily conjugate Quercetin with a suitable saccharide to provide other glycosylated Quercetin for treatment of EC cell hyperplasia-related diseases.

In another embodiment, the present invention is a pharmaceutical formulation may be prepared in accordance with any of the conventional procedures. In preparing the formulation, the active ingredient is admixed or diluted with a pharmaceutical acceptable carrier, or enclosed within a pharmaceutical acceptable carrier which may be in the form of a capsule, sachet or other container. When the carrier serves as a diluent, it may be a solid, semi-solid or liquid material acting as a vehicle, excipient or medium for the Quercetin, glycosylated Quercetin, or mixture thereof. Thus, the formulations may be in the form of a tablet, pill, power, sachet, elixir, suspension, emulsion, solution, syrup, aerosol, soft and hard gelatin capsule, sterile injectable solution, sterile packaged powder and the like.

In a further embodiment, the composition comprises 0.01 to 100 wt % of Quercetin, glycosylated Quercetin, or mixture thereof. Further, the pharmaceutical composition of the present invention can be administered via various routes including oral, transdermal, subcutaneous, intravenous and intramuscular introduction. However, it should be understood that the amount of the Quercetin, glycosylated Quercetin, or mixture thereof would readily be apparent to one skilled in the art in light of various relevant factors including the condition to be treated, the chosen route of administration, the age, sex and body weight of the individual patient, and the severity of the patient's symptom; and therefore, the above dose should not be intended to limit the scope of the invention in any way.

Moreover, in yet another embodiment, the composition is advantageously incorporated in foods or, beverages for the purpose of treating or preventing enterochromaffin cell related diseases, such as inflammatory bowel disease, irritable bowel syndrome, and irritable pouch syndrome.

As described above, it is an embodiment, the present invention can be used as an effective, non-side effect pharmaceutical agent for treating or preventing enterochromaffin cell hyperplasia-related disease such as inflammatory bowel disease, irritable bowel syndrome, and irritable pouch syndrome.

The following examples are intended to further illustrate the present invention without limiting its scope.

### Example 1

### Analgesic Effect of Quercetin in Post-Infectious Irritable Bowel Syndrome (PI-IBS) Rats

1—Induction of PI-IBS Rat Model

Sprague-Dawley male rats (Laboratory Animal Services Centre, The Chinese University of Hong Kong) aged 6 weeks with body weight around 220 g are used to develop PI-IBS model. Briefly, rats are fasted for 24 h, and deeply anesthetized with chloral hydrate (350 mg.kg⁻¹, i.p.). After induction of colitis by 2,4,6-trinitrobenzenesulfonic acid solution (TNBS, 5 mg/0.8 ml/rat, in 50% ethanol, 8 cm from anus) administration, the rats are allowed to recover for at least 4 weeks before use. The control rats are similarly administered with 0.8 ml saline instead of TNBS. Only the rats that have completely recovered from the initial inflammation and acquired visceral hypersensitivity are used as the PI-IBS rats.

2—Study Design and Quercetin Administration

6 groups of 12 rats are tested. Normal rats (Group 1) are treated with water. PI-IBS rats are randomly divided into 5 groups and orally treated with water (Group 2), Doxantrazole (mast cell stabilizer, 30 mg/kg/d, Group 3), and Quercetin (Group 4-6) at the dose of 5, 10, and 20 mg/kg/d, respectively. After 2 weeks daily drug administration, 5 rats in each group are used to measure pain threshold pressure by abdominal withdrawal reflex (AWR) test, and the remaining rat in each test group is used for electromyography (EMG) recording.

3—Evaluate Analgesic Effects of Quercetin

AWR test is performed as previously described in Al-Chaer E D, Kawasaki M, Pasricha P J. A new model of chronic visceral hypersensitivity in adult rats induced by colon irritation during postnatal development. Gastroenterology. 2000 November; 119(5):1276-85. Briefly, rats are lightly anesthetized with ether; a latex balloon is inserted into the descending colon. Rats are then allowed to recover for about 1 h. The tube of the balloon is connected via a Y-connector to a sphygmomanometer and colorectal distension is applied in increments of 5 mmHg until a visible contraction of the abdominal wall is observed by an investigator blinded to the treatment. The pain threshold pressure is defined as the intensity of colorectal distension (CRD) that elicited an observable AWR, i.e. a sudden and persistent abdominal muscle contraction with abdomen lift off the platform. The pain threshold pressure of all groups is recorded and repeated five times with intervals of at least 5 minutes for recovery. EMG recording are conducted as previously described in Tammpere A, Brusberg M, Axenborg J, Hirsch I, Larsson H, Lindstrom E. Evaluation of pseudo-affective responses to noxious colorectal distension in rats by manometric recordings. Pain. 2005 August; 116(3):220-6 and Li Z, Zhang X J, Xu H X, Sung J J, Bian Z X. Intracolonical administration of protease-activated receptor-2 agonists produced visceral hyperalgesia by up-regulating serotonin in the colon of rats. Eur J. Pharmacol. 2009 Mar. 15; 606(1-3):199-204. Firstly, a surgical operation is made to implant the electrodes into the left external abdominal oblique muscles 7 days before EMG recording. After the last drug administration, a flexible latex balloon is inserted into the rat descending colon, and the rats are allowed to accommodate for 1 h. CRD is initiated, and the EMG signal is amplified and filtered by Power Lab System. Graded colorectal distension (20, 40, 60, and 80 mmHg; 20 s duration; 2 min inter-stimulus interval) is carried out for total three cycles to each rat. The changes of area under the curve (AUC) are calculated using that during 20 s distension period over the 20 s baseline. The results are shown in Table 2 and Table 3.

TABLE 2
Effects of Quercetin on visceral pain
threshold pressure in PI-IBS rats

| | | Pain threshold pressure/mmHg | |
|---|---|---|---|
| Groups | Treatments | Before treatment | After treatment |
| (1) Normal control | Water, i.g. | 38 ± 1.5 | 39 ± 1.1 |
| (2) PI-IBS | Water, i.g. | 25 ± 1.8 # | 26 ± 1.9 # |
| (3) Doxantrazole | 30 mg/kg, i.g. | 25 ± 1.3 | 29 ± 1.2 * |
| (4) Quercetin | 5 mg/kg, i.g. | 25 ± 1.9 | 32 ± 1.9 ** |
| (5) Quercetin | 10 mg/kg, i.g. | 25 ± 1.1 | 33 ± 1.8 ** |
| (6) Quercetin | 20 mg/kg, i.g. | 24 ± 1.1 | 35 ± 1.3 ** |

P < 0.05,
** P < 0.01 vs the value before treatment in the same group,
# P < 0.05 vs normal control.

TABLE 3
Effects of Quercetin on visceral motor response to CRD in PI-IBS rats

| | | AUC value under graded distension pressures | | | |
|---|---|---|---|---|---|
| Groups | Treatments | 20 mmHg | 40 mmHg | 60 mmHg | 80 mmHg |
| (1) Normal control | Water, i.g. | 67.40 ± 38.05 | 275.19 ± 49.46 | 496.51 ± 65.20 | 672.87 ± 89.12 |
| (2) PI-IBS | Water, i.g. | 245.20 ± 71.75** | 514.54 ± 56.42** | 707.16 ± 103.51** | 983.76 ± 73.71** |
| (3) Quercetin | 5 mg/kg, i.g. | 161.22 ± 41.53# | 463.55 ± 12.42# | 586.42 ± 56.68# | 906.84 ± 41.08# |
| (4) Quercetin | 10 mg/kg, i.g. | 124.61 ± 67.68# | 386.85 ± 65.68# | 530.98 ± 21.08# | 842.34 ± 62.92# |
| (5) Quercetin | 20 mg/kg, i.g. | 88.28 ± 43.43# | 319.78 ± 118.03# | 470.73 ± 173.52# | 725.28 ± 192.44# |

**P < 0.01 vs normal control,
#P < 0.05 vs PI-IBS rats

As shown in Table 2, pain threshold pressure in PI-IBS rats decreases significantly compared to that of the normal control (p<0.05), demonstrating the PI-IBS rats are characterized with visceral hyperalgesia. The treatment of mast cell stabilizer, Doxantrazole, also significantly attenuates the visceral hyperalgesia in PI-IBS rats (p<0.05). Quercetin treatment dose-dependently elevates the pain threshold pressure when compared to that of the PI-IBS rats (p<0.05), demonstrating Quercetin has analgesic effect in PI-IBS rats. Consistent with the findings from AWR test, the results from EMG recording (Table 3) also show that visceral motor response to graded CRD in PI-IBS rats increases significantly when compared to that of the control (p<0.05). After Quercetin treatment, the visceral motor activity in PI-IBS rats decreases markedly in a dose-dependent manner (p<0.05), indicating the analgesic effect of Quercetin in PI-IBS rats.

### Example 2

### Effect of Quercetin on Colonic EC Cells Number and 5-HT Content in PI-IBS Rats

1—Study Design and Quercetin Administration

Six groups of 5 rats are tested. The groups setting and treatments are as described in Example 1. Different from Example 1, AWR test are not applied to the rats in this experiment in order to obtain the basal data without CRD stimulus. After the last drug administration, a 6 cm proximal colon (1-2 cm from caecum) is collected and divided into 3 parts, the proximal is fixed in 4% paraformaldehyde and embedded in paraffin for EC cell staining; the middle part is collected for 5-HT content measurement and electron microscopic evaluation, and the distal part is for western blotting analysis.

2—EC Cell Counting

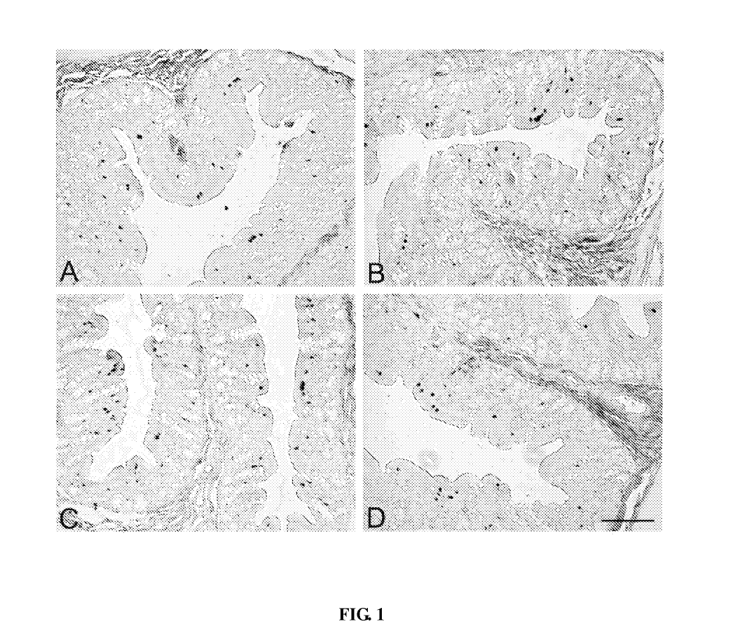

Tissue sections (5-μm thick) are deparaffinized and rehydrated for silver staining. Briefly, sections are incubated with 5% ammoniacal silver solution for 4 h at room temperature, then 2 h in 56° C. and subsequently 12 h at room temperature in a dark humidified chamber. After rinsing with water, 5% sodium hyposulfite is added and the tissue sections are incubated for 5 min at room temperature. The brown to black silver precipitate in the cytoplasm of EC cells represents as a positive reaction. A researcher blinded to treatment counted 5 random fields at 200× magnifications for each section, and the number of EC cells per mm² of mucosa is quantified using Image J NIH software. The results are shown in Table 4, and the representative EC cells staining are shown in FIG. 1.

3—5-HT Content Assessment

5-HT content in the colon tissue is assayed based on our previously reported procedure in Qi S D, Tian S L, Xu H X, Sung J J, Bian Z X. Quantification of luminally released serotonin in rat proximal colon by capillary electrophoresis with laser-induced fluorescence detection. Anal Bioanal Chem. 2009 April; 393(8):2059-66. Briefly, the colon segment is homogenized in 15% iced trichloroacetic acid; for 2 min and stored at 4° C. for 2 h. After centrifuge at 10,000 G for 15 min, the supernatant of each sample is filtered using 0.22 μm filters and extracted with diethyl ether for five times to remove the trichloroacetic acid. The prepared samples are added to derivatization solution (150 mM sodium tetraborate, 25% acetonitrile, 2 mM CFSE) and incubated for 30 mM in dark at room temperature. The 5-HT content is analyzed by capillary electrophoresis with laser-induced fluorescence detection and expressed as nanogram per milligram (wet weight of tissue). The results are shown in Table 4.

TABLE 4
Effect of Quercetin on colonic EC cells
number and 5-HT content in PI-IBS rats

| | | EC cell density | 5-HT content |
|---|---|---|---|
| Groups | Treatments | (Number/mm²) | (ng/mg) |
| (1) Normal control | Water, i.g. | 68 ± 12 | 4.40 ± 0.73 |
| (2) PI-IBS | Water, i.g. | 101 ± 6 * | 6.35 ± 0.87 * |
| (3) Doxantrazole | 30 mg/kg, i.g. | 95 ± 10 | 5.75 ± 0.44 |
| (4) Quercetin | 5 mg/kg, i.g. | 89 ± 5 # | 5.15 ± 0.58 * |
| (5) Quercetin | 10 mg/kg, i.g. | 72 ± 7 # | 4.63 ± 0.92 * |
| (6) Quercetin | 20 mg/kg, i.g. | 105 ± 8 | 5.50 ± 0.65 |

P < 0.05 vs normal control,
# P < 0.05 vs PI-IBS rats °

As shown in Table 4, the colonic EC cells number and 5-HT content are significantly increased in PI-IBS rats (˜48% in EC cells and ˜23% in 5-HT content) when compared to that of the control (p<0.05), suggesting the occurrence of colonic EC cells hyperplasia in PI-IBS rats. Compared with the PI-IBS group, Low and median dose Quercetin treatment markedly decreased the EC cells number (18% and 42%) and 5-HT content (28% and 35%) in PI-IBS rats, demonstrating the activity of Quercetin in reducing EC cells hyperplasia in PI-IBS rats. However, mast cell stabilizer Doxantrazole treatment shows no marked effects on colonic EC cells number and 5-HT content in PI-IBS rats.

### Example 3

### Effect of Quercetin on Colonic TPH Expression in PI-IBS Rats

The colon tissues are homogenized and sonicated on ice for protein extraction. After determination of protein content, the samples protein are denatured at 100° C. for 5 min. Proteins are separated by 10% SDS-PAGE and transferred to PVDF membranes (Bio-Rad, CA). After blocking with 5% nonfat milk, the membranes are incubated overnight at 4° C. with rabbit anti-TPH antibody (1:1000, Santa Cruz, Calif.), then incubated with the appropriate secondary antibodies for 1 h at room temperature. The immunoreaction is detected using ECL Western blotting kit (ECL, Amersham, UK). Bands are visualized on Biomax X-ray film, and optical density of each band is semi-quantified by computer software (Image J.NIH). All detected protein bands are normalized to β-actin levels. Data are shown in Table 5.

TABLE 5
Effect of Quercetin on colonic TPH expression in PI-IBS rats

| Groups | Treatments | Sample number | TPH/actin ratio |
|---|---|---|---|
| (1) Normal control | Water, i.g. | 4 | 0.55 ± 0.12 |
| (2) PI-IBS | Water, i.g. | 4 | 0.80 ± 0.10 * |
| (3) Quercetin | 10 mg/kg, i.g. | 4 | 0.62 ± 0.11 # |

P < 0.05 vs normal control,
# P < 0.05 vs PI-IBS rats.

Concerning the important role of the rate-limiting enzyme in 5-HT synthesis, colonic TPH expression is further evaluated by Western blot technique. As shown in Table 5, TPH protein expression in PI-IBS rats significantly increases (˜31%) when compared to that of the control (p<0.05), while median dose Quercetin (10 mg/kg) treatment markedly reduces colonic TPH expression in PI-IBS rats (˜22.8%, p<0.05).

### Example 4

### Effect of Quercetin on Mechanical Stimuli-Induced 5-HT Release in PI-IBS Rats

1—Study Design and 5-HT Content Determination

To determine whether mechanical stimuli (CRD) can induce colonic 5-HT release in PI-IBS rats, and whether Quercetin has effect on it, 5-HT content in colonic tissue is evaluated in rats with or without AWR test. The colon tissues for 5-HT content determination are collected from the rats from Example 1 and Example 2, and the methods for 5-HT content determination are followed as described in Example 2. Results are shown in Table 6.

2—Electron Microscopy Evaluation

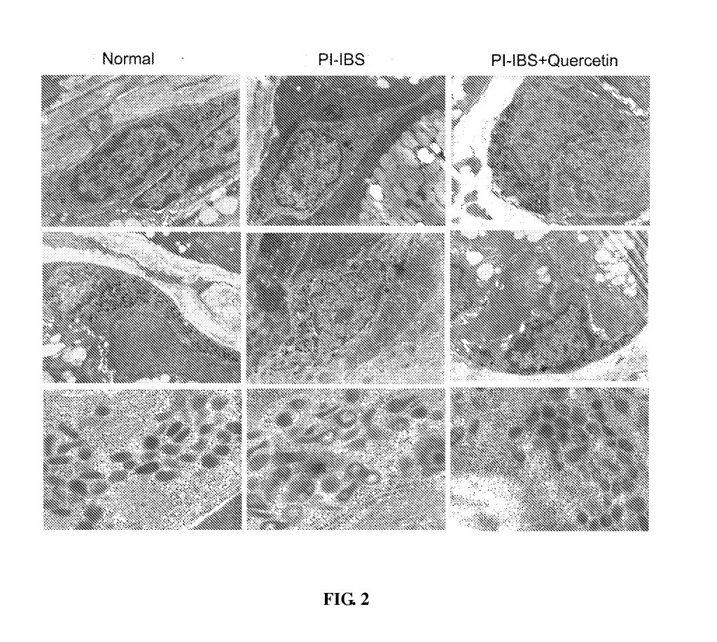

For electron microscopy evaluation, the colon tissues are collected from the rats treated with AWR test so as to investigate the feature of mechanical stimulus-induced 5-HT release. Colon segment is fixed for 24 h at 4° C. in fixative containing 4% paraformaldehyde and 0.2% picric acid in 0.1M PB, then washed and embedded in gelatin. Thin sections are cut and picked up on 200-mesh grids. The sections are stained with uranyl acetate and lead citrate. Examination and photography of sections are carried out using Philips CM 10 transmission electron microscope (Philips Scientifics, Netherlands). At the ultra-structural level, EC cell are distinguished from other enteroendocrine cells by the pleomorphic nature of their secretory granules. The EC cells in which there are many clear secretory granules without cores and granules with eccentric cores are considered as the activated one with excessive 5-HT release. The representative photos are shown in FIG. 2.

TABLE 6
Effect of Quercetin on mechanical stimuli-
induced 5-HT release in PI-IBS rats

| | | 5-HT content of tissue | |
|---|---|---|---|
| Groups | Treatments | −CRD | +CRD |
| (1) Normal control | Water, i.g. | 4.40 ± 0.73 | 4.12 ± 1.04 |
| (2) PI-IBS | Water, i.g. | 6.35 ± 0.87 * | 2.96 ± 0.29 § |
| (3) Doxantrazole | 30 mg/kg, i.g. | 5.75 ± 0.44 | 2.62 ± 0.30 § |
| (4) Quercetin | 5 mg/kg, i.g. | 5.15 ± 0.58 # | 4.96 ± 0.91 |
| (5) Quercetin | 10 mg/kg, i.g. | 4.63 ± 0.92 # | 4.29 ± 0.58 |
| (6) Quercetin | 20 mg/kg, i.g. | 5.50 ± 0.65 | 4.01 ± 1.14 § |

P < 0.05 vs normal control,
# P < 0.05 vs PI-IBS rats,
§ P < 0.05 vs that after CRD application.

As shown in Table 6, after CRD applied, the tissue 5-HT content in normal rats reduced by ˜17%, but there are no statistical differences when compared to that without CRD; while in PI-IBS rats, tissue 5-HT content dramatically decreased by ˜68% after CRD application (p<0.01). In Doxantrazole treated rats, 5-HT content in colon tissue markedly decreases after CRD application (˜54.4%, p<0.05). After low and median dose of Quercetin treatment, 5-HT content are both decreased after CRD application (˜3.67% and ˜7.34%), but no significant difference is found when compared to that without CRD. Results from electron microsgraphs also showed that, compared to that of the control group, the color of secretory granules in EC cells of PI-IBS rats are reduced with many cores and empty vesicles after CRD application, demonstrating excessive 5-HT is released after mechanical stimulation. In median dose of Quercetin treated group, the secretory granules became denser when compared to that of the PI-IBS rats. All these data illustrate that low and median dose of Quercetin treatment also attenuate CRD-induced excessive 5-HT release in PI-IBS rats.

### Example 5

### Effect of Quercetin on Pain Threshold Pressure, Colonic EC Cell Number and 5-HT Content in Normal Rats

To identify whether the effects of Quercerin in PI-IBS rats are specific, pain threshold pressure, colonic EC cell number and 5-HT content are further evaluated in normal rats treated with Quercetin. Briefly, Quercetin at the dose of 10 mg/kg is administered to normal rats orally for 14 days. After the final drug administration, pain threshold pressure, colonic EC cell density and 5-HT content are evaluated and the methods used here are as same as that shown in above experiments.

TABLE 7
Effects of Quercetin on pain threshold pressure, colonic
EC cell density and 5-HT content in normal rats

| | | Pain threshold | EC cell density | 5-HT content |
|---|---|---|---|---|
| Groups | Treatments | pressure (mmHg) | (Number/mm²) | (ng/mg) |
| (1) Normal control | Water, i.g. | 38.4 ± 1.4 | 90 ± 5 | 4.12 ± 1.04 |
| (2) Quercetin | 10 mg/kg, i.g. | 37.9 ± 1.7 | 91 ± 10 | 4.06 ± 0.48 |

As shown in Table 7, compare with the normal rats treated with water, there is no significant differences in pain threshold pressure, colonic EC cell density and 5-HT content between normal rats treated with Quercerin and water. These results indicated that Quercetin can attenuate visceral hyperalgesia, reduce colocic EC cell hyperplasia and 5-HT content in PI-IBS rats, but has little effects in normal rats.

### Example 6

### Effects of Quercetin on Cell Viability in QGP-1 Cells

1—Cell Culture and Study Design

To investigate the underlying mechanisms of Quercein on EC cell hyperplasia, a EC cell model, QGP-1 cell line is used as EC cell model. Human pancreatic carcinoma QGP-1 cells (JCRB0183, HSRRB, Japan) are cultured in RPMI 1640 supplemented with 10% fetal bovine serum, 100 IU/ml penicillin, and 100 μg/ml streptomycin in a humidified atmosphere of 95% air and 5% CO₂ at 37° C. Cells are passaged using 0.025% trypsin and 0.01% EDTA. Cells are trypsinized and seeded in 96-well culture dishes at a density of 5,000-8,000 cells/well and cultured for 24 h or 48 h in the presence of Quercetin (0-100 μM, 0.01% DMSO).

2—Measurement of Cell Viability (MTT Assay)

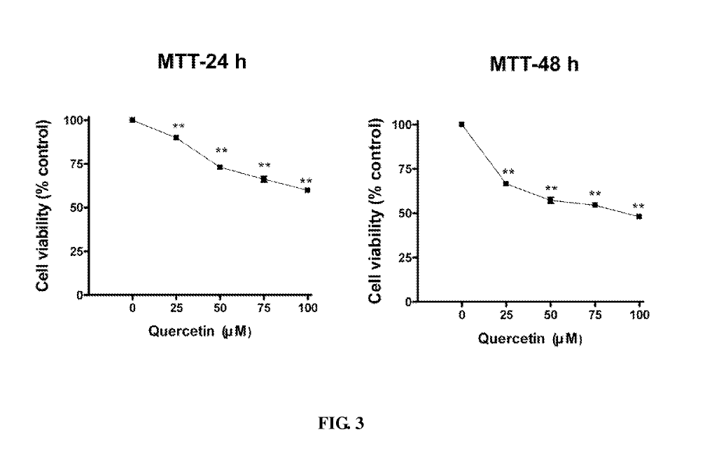

3-(4,5-dimethylthiazol-2yl-)-2,5-diphenyl tetrazolium bromide (MTT) is used to evaluate cell viability. In this assay, cells are seeded in 96-well dishes and cultured for 2 in the presence of Quercetin. Subsequently, the culture medium is replaced with 100 μl/well fresh medium containing 0.5 mg/ml MTT, and the cells are further incubated for 4 h at 37° C. The reaction is stopped by adding 100 μl/well 10% SDS in 0.1 M HCl, the absorbance of the plates is read at 570 nm The percent of cell survival is calculated by comparing the average absorbance of the Quercetin treated cells with the corresponding absorbance of the DMSO treated cells. All doses are tested in triplicates and repeated for at least three times. The results are shown in FIG. 3.

As shown in FIG. 3, the cells treated with Quercetin (25-100 μM) for 24 h shows a significantly decreased cell survival in a dose-dependent manner, and the ˜50% reduction of cell viability in comparison with the control is achieved at the dose about 100 μM. After 48 h incubation with Quercetin, cell viability dramatically and dose-dependently decreased, and the IC50 is about 50 μM. In contrast to Quercetin treated cells, the cells treated with DMSO show little cytotoxicity.

### Example 7

### Cytotoxicity of Quercetin on QGP-1 Cells

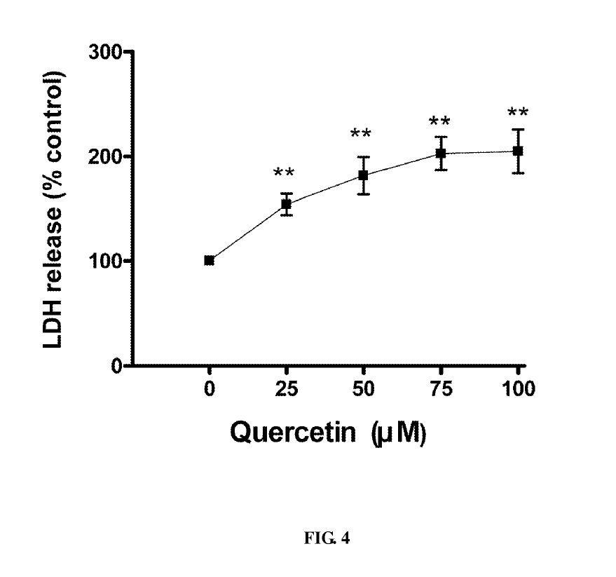

The cytotoxicity of Quercetin is determined by lactate dehydrogenase (LDH) assay in QGP-1 cells. Cells are seeded at a density of 5,000 cells/well into 96-well plates and incubated with Quercetin (0-100 μM, 0.01% DMSO) for 24 h. Release of cytoplasmic enzyme lactate dehydrogenase is measured after incubation by using colorimetric assay kit (Roche Molecular Biochemicals, Germany) according to the manufacturer's instructions. The data are shown in FIG. 4.

As LDH release mainly occur under the condition of cell membrane damage, a quantitative cytotoxicity can be determined by measuring the LDH activity released from the damaged cells. As shown in FIG. 4, incubation QGP-1 cells with Quercetin (25-100 μM) for 24 h results in a significant and dose-dependent increase in LDH release. These data suggested that Quercetin (25-100 μM) is cytotoxic against QGP-1 cells.

### Example 8

### Effects of Quercetin on Cell Cycle Modulation in QGP-1 Cells

1—Flow Cytomethic Ananlysis

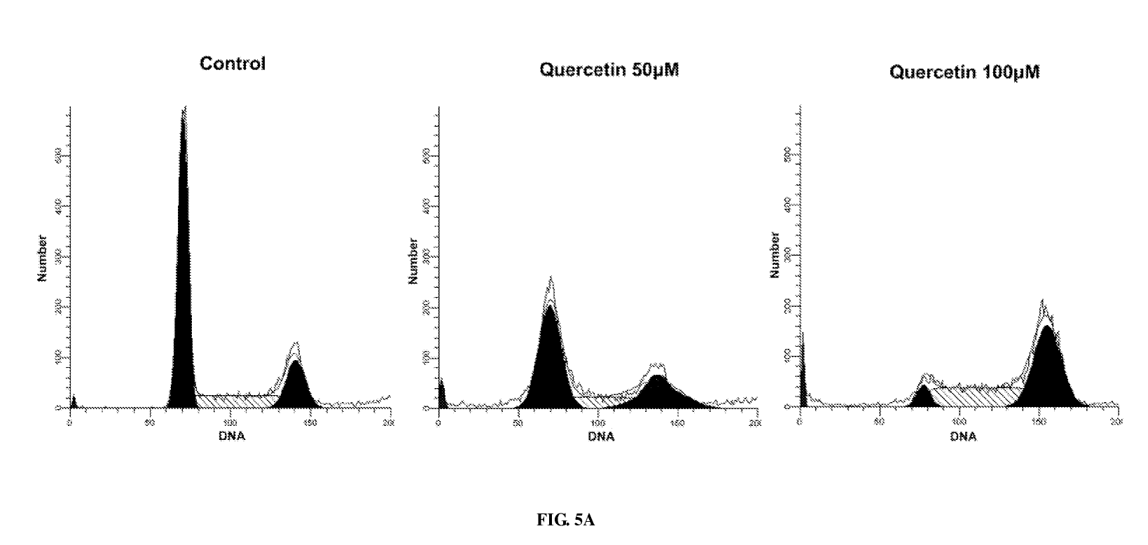

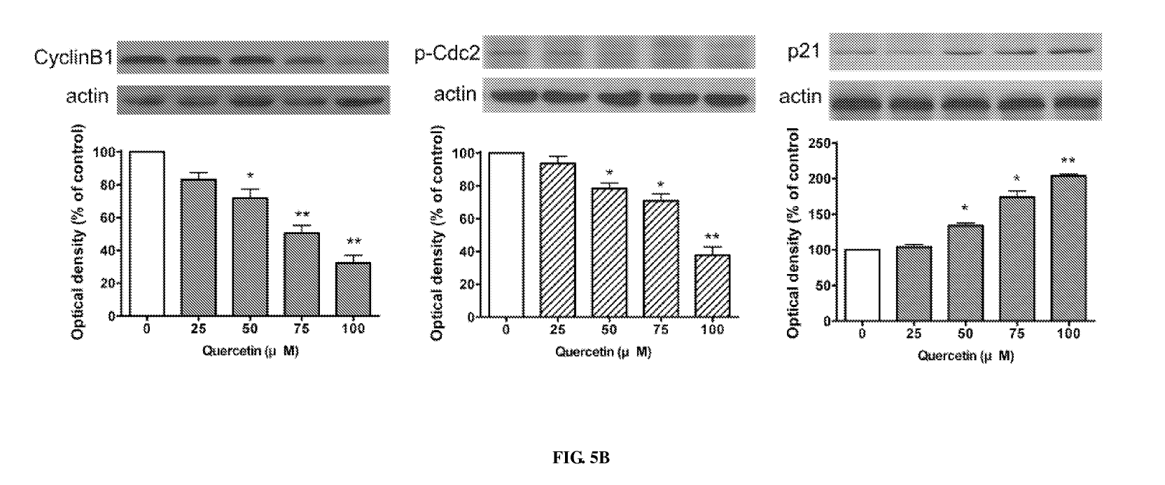

QGP-1 cells are seeded at a density of 10×10⁴ cells/ml and pre-incubated overnight. After incubation with or without Quercetin (0-100 μM) for 24 h, cells are harvested by trypsinization and fixed in 70% ethanol at 4° C. overnight. The cells are centrifuged to remove ethanol and washed twice using ice-cold PBS. The cell pallets are resuspended in 500 n1 propidium iodide solution (0.1% Triton X-100, 50 μg/ml propidium iodide in PBS) containing 50 μg/ml RNase and incubated at 37° C. for 30 min and protected from the light. The DNA content of 10,000 cells is detected by BD FASCanto Flow Cytometry and analyzed using CellFit software (Becton Dickinson, Heidelberg, Germany). The data are representative of those obtained in at least three experiments. The results are shown in Table 8 and FIG. 5A.

2—Western Blot Analysis

After drug treatment, the cells are lysed and centrifuged to collect the protein extract After determination of protein content, the samples protein are denatured at 100° C. for 5 min Proteins are separated by 10-12% SDS-PAGE and transferred to PVDF membranes (Bio-Rad, CA). After blocking with 5% nonfat milk for 1 h at room temperature, the membranes are incubated overnight at 4° C. with primary antibodies (mouse anti-cyclin BE 1:500, Abcam; goat anti-p-Cdc2:1:500, Santa Cruz; mouse anti-p21: 1:500, BD Bioscience). After washing three times in TBST, the membranes where incubated with the appropriate secondary antibodies (1:2000, Invitrogen) for 1 h at room temperature. The immunoreaction is detected using ECL Western blotting kit (ECL, Amersham, UK). Bands are visualized on Biomax X-ray film, and optical density of each band is semi-quantified by software (Image J.NIH). In order to adjust for any variations in loading, all detected protein bands are normalized to β-actin levels. Western immunoblots and statistical analysis results are shown in FIG. 5B.

TABLE 8
Cell cycle analysis of QGP-1 cells after Quercetin treatment

| Cell cycle | Quercetin (μM) | | | |
|---|---|---|---|---|
| phases | 0 (%) | 25 (%) | 50 (%) | 100 (%) |
| G0/G1 | 57.6 ± 2.4 | 56.0 ± 3.5 | 47.8 ± 4.0 * | 8.4 ± 1.7 ** |
| S | 22.9 ± 0.8 | 22.3 ± 0.3 | 32.0 ± 0.4 * | 43.9 ± 0.7 ** |
| G2/M | 19.4 ± 1.8 | 21.8 ± 3.2 | 20.2 ± 4.29 | 47.7 ± 2.2 ** |

p < 0.05,
**p < 0.01 vs. the control

As shown in Table 8, the cell cycle analysis shows that treatment of cells for 24 h with Querctin (50 and 100 μM) concentration-dependently increased the percentage of cells in G2/M phase, accompanied with the decreased percentage of cells in G1 phases. The result demonstrates that Querctin inhibits QGP-1 cell proliferation by inducing G2/M phase cell cycle arrest. Knowing that G2/M phase transition is involving Cdc-2 activation, activated Cdc-2 binding to cyclin B 1, and synthesized cyclin B 1, the protein expression of cyclin B1 and p-Cdc-2 in QGP-1 cells are further examined at 24 hours after Quercetin treatment. The protein levels of cyclin B1 and p-Cdc-2 all significantly decrease after 50-100 μM Quercetin treatment P21 has been considered as an important regulator for G2/M arrest in mammalian cells, p21 protein expression in Quercetin-treated QGp-1 cells is further evaluated. Our results showed that the protein levels of p21 are significantly increased after 50-100 nM Quercetin treatment This effect of Quercetin is consistent with the protein alteration of cyclin B1 and p-Cdc-2 Therefore, p21 mediated molecular pathway is involved in Quercetin-induced G2/M cell phase arrest by reducing cyclin B1 and p-Cdc-2 protein expression. The representative flow cytometric figures and the altered proteins expression are shown in FIG. 5.

### Example 9

### Effects of Quercetin on Cell Apoptosis in QGP-1 Cells

1—Fluorescence Microscopy Analysis

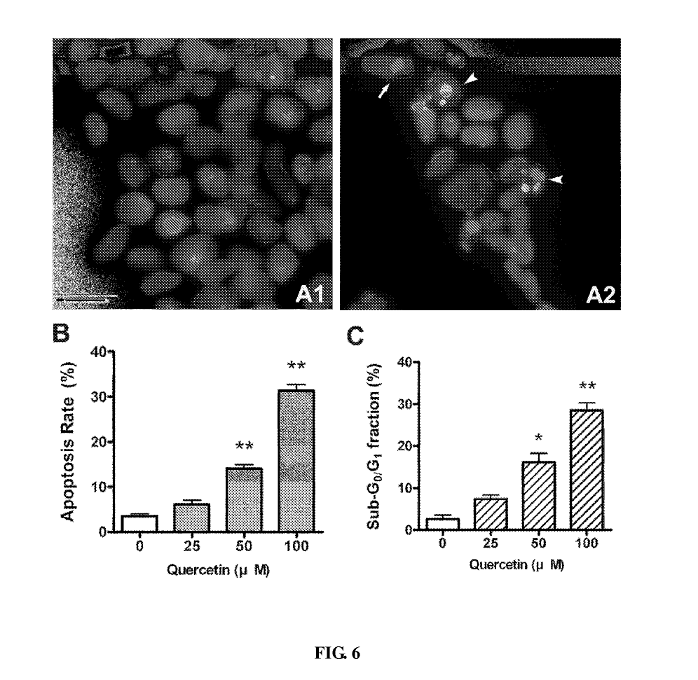

Cells are seeded on coverslips in 24-well plate at the density of 100,000 cells per well and cultured overnight. After exposure to DMSO (control) or different concentration of Quercetin for 24 h, cells are washed three times and fixed with 4% paraformaldehyde for 20 min at room temperature. After washing in PBS, cells are permeabilized in 0.2% tritonX-100 for 10 min and stained with DAPI (20 μg/ml) for 10 min at room temperature in darkness. Slides are examined with a laser scanning microscope (Olympus Fluoview FV1000) 40× magnification. Three hundred cells are counted per slide randomly as previously reported in Ramage L, Jones A C, Whelan C J. Induction of apoptosis with tobacco smoke and related products in A549 lung epithelial cells in vitro. J Inflamm (Lond). 2006; 3:3. Apoptosis cells condense or fragment nuclear chromatin with or without apoptotic bodies, while normal cells have typical morphology with smooth nuclear and membrane. The representative figures and results are shown in FIG. 6A and FIG. 6B.

2—Flow Cytomethic Analysis

For flow cytometric analysis, cells with sub-G0/G1 DNA content are stained with propidium iodide, and the methods are as described in section 2 of Example 8. The results are shown in FIG. 6C. As shown in FIG. 6, apoptosis rate and sub-G0/G1 fraction concentration-dependently increases in QGP-1 cells treated with 25˜100 μM Quercetin. Based on the results form LDH assay and apoptosis detection, Quercetin indeed has cytotoxicity and can further induce cell apoptosis.

### INDUSTRIAL APPLICABILITY

The present invention discloses a method of treating or preventing enterochromaffin cell hyperplasia-related diseases in a mammal. In particular, the present invention relates to a method of preventing or treating enterochromaffin cell hyperplasia-related diseases such as inflammatory bowel disease, irritable bowel syndrome and irritable pouch syndrome in mammals, which comprises by administering an effective amount of Quercetin.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A method for treatment or prevention of enterochromaffin cell hyperplasia-related diseases comprising administering a composition comprising an effective amount of Quercetin, glycosylated Quercetin or a mixture thereof, wherein the enterochromaffin cell hyperplasia-related diseases are induced by infection or develop after infection.

2. The method according to claim 1 wherein the enterochromaffin cell hyperplasia-related diseases comprising inflammatory bowel disease, irritable bowel syndrome, irritable pouch syndrome, post-infectious inflammatory bowel disease, post-infectious irritable bowel syndrome, post-infectious irritable pouch syndrome, untreated coeliac diseases, chronic gastritis of pernicious anaemia, hypergastrinemia-related diseases and gastrointestinal disorders induced by infections.

3. The method according to claim 1 wherein the enterochromaffin cell hyperplasia-related diseases occur in mammals.

4. The method according to claim 1 further comprises incorporating said composition into foods or beverages.

5. The method according to claim 1 further comprises formulating said composition into a pharmaceutical acceptable formulation by admixing or diluting said composition with a pharmaceutically acceptable carrier.

6. The method according to claim 5, wherein said pharmaceutically acceptable carrier is a solid, semi-solid or liquid material acting as a vehicle, excipient or medium for said composition.

7. The method according to claim 1, further comprises formulating said composition into a pharmaceutical acceptable formulation by enclosing said composition within a pharmaceutical acceptable carrier.

8. The method according to claim 7, wherein said carrier is a capsule, sachet or other container.

9. The method according to claim 5 wherein the pharmaceutical acceptable formulation is in the form of a tablet, pill, power, sachet, elixir, suspension, emulsion, solution, syrup, aerosol, soft and hard gelatin capsule, sterile injectable solution, sterile packaged powder.

10. The method according to claim 9 comprises administering said pharmaceutical formulation via route comprising oral, transdermal, subcutaneous, intravenous or intramuscular introduction.

11. The method according to claim 10 wherein the Quercetin, glycosylated Quercetin or mixture thereof is in an amount ranging from 0.01 to 100 wt % depending on factors comprising condition to be treated, chosen route of administration, age, sex and body weight of an individual patient, and severity of the patient's symptom.

12. A composition for treatment of enterochromaffin cell hyperplasia-related diseases comprising Quercetin, glycosylated Quercetin or a mixture thereof, wherein the enterochromaffin cell hyperplasia-related diseases are induced by infection or develop after infection.

13. The composition of claim 12, wherein said composition is administered orally, transdermally, subcutaneously, intravenously and intramuscularly.

14. The composition of claim 12, wherein the composition is incorporated in foods or beverages.

15. The composition of claim 12, wherein the Quercetin glycosylated Quercetin or a mixture thereof is in the amount of 0.01-100% wt.

16. A composition for prevention of enterochromaffin cell hyperplasia-related diseases comprising Quercetin, glycosylated Quercetin or a mixture thereof, wherein the enterochromaffin cell hyperplasia-related diseases are induced by infection or develop after infection.

17. The composition of claim 16, wherein said composition is administered orally, transdermally, subcutaneously, intravenously and intramuscularly.

18. The composition of claim 16, wherein the composition is incorporated in foods or beverages.

19. The composition of claim 16, wherein the Quercetin glycosylated Quercetin or a mixture thereof is in the amount of 0.01-100% wt.
